(19) United States
(12) Patent Application Publication
(10) Pub. No.: US 2019/0200554 A1
(43) Pub. Date: Jul. 4, 2019

(54) Compositions and Methods for Plant Haploid Induction

(71) Applicant: Monsanto Technology LLC, St. Louis, MO (US)

(72) Inventors: Rico A. Caldo, Eureka, MO (US); Paul S. Chomet, Groton, CT (US); Rahul Dhawan, Ballwin, MO (US); Yan Fu, Chesterfield, MO (US); Jonathan Lamb, Wildwood, MO (US); Bryce M. Lemke, Nevada, IA (US); Jeanette M. Peevers, Chesterfield, MO (US)

(21) Appl. No.: 16/325,832

(22) PCT Filed: Aug. 15, 2017
(86) PCT No.: PCT/US17/46837

§ 371 Date: Feb. 15, 2019

Related U.S. Application Data
(60) Provisional application No. 62/375,618, filed on Aug. 16, 2016.

(51) Int. Cl. A01H 1/08

(52) U.S. Cl. CPC A01H 1/08

(57) ABSTRACT

The present invention provides compositions and methods for producing haploid induction. Genetic elements associated with haploid induction, recombinant DNA constructs comprising the genetic elements are also provided. Methods are further provided for generating haploid inducer plants, haploid plants, and doubled haploid plants (including spontaneous diploidization).

### CROSS-REFERENCE TO RELATED APPLICATION

This application claims priority benefit to U.S. Provisional Patent Application No. 62/375,618, filed on Aug. 16, 2016, which is incorporated herein by reference in its entirety.

### INCORPORATION OF SEQUENCE LISTING

A computer readable form of a sequence listing is filed with this application by electronic submission and is incorporated into this application by reference in its entirety. The sequence listing is contained in the file named “62110WO_Sequence_Listing_ST25.txt”, which is 1,536,569 bytes in size (measured in operating system MS Windows) and created on Aug. 16, 2016.

### FIELD OF THE INVENTION

The present invention relates to compositions and methods for producing haploid inducer plants, haploid plants, and doubled haploid plants (including the spontaneous diploidization).

### BACKGROUND

Plant breeding is greatly facilitated by the use of doubled haploid (DH) plants. The production of doubled haploid plants enables plant breeders to obtain inbred lines without multi-generational inbreeding, thus decreasing the time required to produce homozygous plants. Doubled haploid plants provide an invaluable tool to plant breeders, particularly for generating inbred lines, QTL mapping, cytoplasmic conversions, and trait introgression. Improvement on haploid production and screening are the key components for a successful implementation of the doubled haploid technology in plant breeding programs. Haploids are traditionally generated through an androgenesis or gynogenesis approach (Hiebert, C. et al., Theor Appl Genet 117: 581-594 (2008)). Some plant species such as maize, Arabidopsis, and barley can produce haploids by uniparental genome elimination via a male inducer. In corn, the haploids are generated spontaneously when crossed to the maize inducer lines. Production of haploids using in vivo induction method has been widely adopted for generating new inbred lines however haploid induction rate remains low.

The molecular mechanisms underlying haploid induction in maize are still unclear. Previous QTL mapping studies for unraveling the genetic architecture of haploid induction detected a major QTL on chromosome 1. The most comprehensive study with four bi-parental populations (Prigge et al., Genetics 190: 781-793 (2012)) mapped this QTL, termed qhir1, to bin 1.04 and hypothesized that it is required for haploid induction, but QTL positions and 1-LOD support intervals differed substantially among populations. In another study with population 1680×UH400, qhir1 was fine-mapped to a 3.57 Mb region between markers umc1917 and bnlg1811, and a 243 kb region was identified with significant effect on haploid induction (Dong et al., Theoretical and Applied Genetics 126: 1713-1720 (2013)). The present invention provides a fine-mapped region (MonI1) that confers maternal haploid induction in maize obtained through high density fine mapping and QTL cloning. The candidate genes and genetic elements associated with haploid induction are also provided for producing haploid inducer plants. The present invention further provides methods for obtaining haploids and doubled haploids (including the spontaneous diploidization) by genetically modifying the presently disclosed candidate genes and genetic elements.

### SUMMARY

In one aspect of the present invention, a recombinant DNA construct is provided comprising a heterologous promoter functional in a plant cell and operably linked to: a polynucleotide that comprises a nucleotide sequence with at least 90%, at least 91%, at least 92%, at least 93%, at least 94%, at least 95%, at least 96%, at least 97%, at least 98%, at least 99% identity, or 100% identity to a sequence selected from the group consisting of SEQ ID NOs: 1-5, and their complements, or a functional fragment thereof; a polynucleotide that comprises a nucleotide sequence with at least 90%, at least 91%, at least 92%, at least 93%, at least 94%, at least 95%, at least 96%, at least 97%, at least 98%, at least 99% identity, or 100% identity to a sequence selected from the group consisting of SEQ ID NOs: 20-24, 86, 107 and 109; a polynucleotide that encodes a polypeptide having an amino acid sequence with at least 90%, at least 91%, at least 92%, at least 93%, at least 94%, at least 95%, at least 96%, at least 97%, at least 98%, at least 99% identity, or 100% identity to a sequence selected from the group consisting of SEQ ID NOs: 42-46, 108 and 110; or a polynucleotide that comprises a nucleotide sequence that suppresses at least one endogenous target gene having an amino acid sequence with at least 90%, at least 91%, at least 92%, at least 93%, at least 94%, at least 95%, at least 96%, at least 97%, at least 98%, at least 99% identity, or 100% identity to a sequence selected from the group consisting of SEQ ID NOs: 89, 90, 92 and 100-106. The synthetics miRNA sequences SEQ ID NOs: 111 and 112 are also provided for suppressing the corn Mon-DKD2 endogenous genes SEQ ID NO: 87 and SEQ ID NO: 88, respectively. The recombinant DNA construct of the present invention may comprise a combination of two or more nucleotide sequences described above.

In another aspect of the present invention, transgenic plants, plant cells, plant tissues and plant parts are further provided comprising an insertion of the recombinant DNA construct of the present invention into the genome of such plants, cells, tissues, and plant parts. Transgenic plants of the present invention exhibit haploid induction phenotype when crossed to a non-inducer line, relative to a control or wild type plant not having the recombinant DNA construct.

In another aspect, the disclosure provides a plant comprising a recombinant DNA construct of the present disclosure, wherein the plant is a progeny, a propagule, or a field crop.

In another aspect, the disclosure provides a field crop comprising a recombinant DNA construct of the present disclosure, wherein the field crop is selected from the group consisting of corn, soybean, sorghum, cotton, canola, rice, barley, oat, wheat, turf grass, alfalfa, sugar beet, sunflower, quinoa and sugar cane.

In another aspect, the disclosure provides a propagule comprising a recombinant DNA construct the present disclosure, wherein the propagule is selected from the group consisting of cell, pollen, ovule, flower, embryo, leaf, root, stem, shoot, meristem, grain and seed.

In another aspect of the present invention, a method for producing a haploid inducer plant is provided comprising (a) transforming at least one cell of an explant with a recombinant DNA construct comprising a nucleotide sequence described above; and (b) regenerating or developing the transgenic plant from the transformed explant. Such methods may further comprise (c) selecting a plant that exhibits haploid induction phenotype when crossed to a non-inducer plant as compared to a control plant not having the recombinant DNA construct.

In another aspect, the disclosure provides a recombinant DNA construct comprising a donor template, wherein said donor template comprises at least one homology arm flanking a recombinant sequence for modulation of expression of an endogenous gene where said gene is encoded by a polynucleotide that comprises a nucleotide sequence with at least 90%, at least 91%, at least 92%, at least 93%, at least 94%, at least 95%, at least 96%, at least 97%, at least 98%, at least 99% identity, or 100% identity to a sequence selected from the group consisting of SEQ ID NOs: 20-24, 86-88, 91, 93-99, 107 and 109; or a polynucleotide that encodes a polypeptide having an amino acid sequence with at least 90%, at least 91%, at least 92%, at least 93%, at least 94%, at least 95%, at least 96%, at least 97%, at least 98%, at least 99% identity, or 100% identity to a sequence selected from the group consisting of SEQ ID NOs: 42-46, 89-90, 92, 100-106, 108 and 110.

In another aspect, the disclosure provides a method for obtaining a haploid inducer plant comprising the steps of identifying an endogenous genomic locus corresponding to a gene selected from the group consisting of SEQ ID NOs: 42-46, 89-90, 92, 100-106, 108 and 110, or its homologs; and site-specifically inserting a recombinant sequence capable of modulating expression of said gene.

In another aspect, the disclosure provides a method for obtaining a haploid inducer plant comprising the steps of: identifying in a non-inducer line the haploid induction region corresponding to the MonI1 regions identified in KHI1; modifying the identified haploid induction region by: deleting the entire or portions of the region; or swapping the entire or portions of the region with the KHI1 haploid induction region; regenerating or developing the transgenic plant comprising the modified haploid induction region in its genome; and selecting a plant that exhibits haploid induction phenotype when crossed to a non-inducer plant.

In another aspect, the present invention provides a method for obtaining a doubled haploid plant comprising the steps of: crossing the transgenic inducer plants with non-inducer plants of interest to produce haploids; and producing doubled haploid plants by chromosome doubling of the haploids.

In another aspect, the present invention provides a method for transferring haploid induction effect to new lines comprising the steps of: providing a first plant comprising at least one supernumerary chromosome, wherein the at least one supernumerary chromosome comprises at least one genetic element that can cause haploid induction; crossing the first plant with a second plant of interest; and recovering a third plant resultant from crossing the first plant and second plant, wherein the third plant comprises at least one supernumerary chromosome comprises at least one genetic element that can cause haploid induction.

### BRIEF DESCRIPTION OF THE SEQUENCE LISTING

SEQ ID NO: 1 is the contiguous DNA sequence formed by the assembly of BAC sequences spanning the extended MonI1 region in corn haploid induction line KHI1.

SEQ ID NOs: 2-4 are the BAC DNA sequences spanning the MonI1 region in KHI1.

SEQ ID NO: 5 is the DNA sequence for the MonI1 region in KHI1.

SEQ ID NO: 6 is the DNA sequence for the MonI1 region in corn B73.

SEQ ID NOs: 7 and 8 are the marker sequences flanking the MonI1 region in B73.

SEQ ID NOs: 9-30 are the coding DNA sequences for genes identified in SEQ ID NO: 1.

SEQ ID NOs: 31-52 are the amino acid sequences encoded by SEQ ID NOs: 9-30, respectively.

SEQ ID NOs: 53-85 are the DNA sequences for the non-coding RNAs identified in SEQ ID NO: 1.

SEQ ID NO: 86 is the genomic DNA sequence corresponding to the patatin-like phospholipase (PNPLA) gene (having the protein sequence of SEQ ID NO: 42) identified in the MonI1 region in KHI1.

SEQ ID NOs: 87 and 88 are the coding DNA sequences for genes identified in the MonI1 region in Mon-DKD2 but not in KHI.

SEQ ID NOs: 89 and 90 are the protein sequences encoded by SEQ ID NOs: 87 and 88, respectively.

SEQ ID NOs: 91 is the coding DNA sequence for the PNPLA gene identified in the MonI1 region in B73. SEQ ID NO: 92 is the protein sequence encoded by SEQ ID NO: 91.

SEQ ID NOs: 93-99 are the coding DNA sequences for genes identified in the MonI1 region in B73 but not in KHI1. SEQ ID NOs: 100-106 are the protein sequence encoded by SEQ ID NOs: 93-99, respectively.

SEQ ID NO: 107 is the coding DNA sequence for the PNPLA gene in rice. SEQ ID NO: 108 is the protein sequence encoded by SEQ ID NO: 107.

SEQ ID NO: 109 is the coding DNA sequence for the PNPLA gene in sorghum. SEQ ID NO: 110 is the protein sequence encoded by SEQ ID NO: 107.

SEQ ID NO: 111 is the synthetic miRNA sequence designed to suppress expression of the target gene (SEQ ID NO: 87) in Mon-DKD2.

SEQ ID NO: 112 is the synthetic miRNA sequence designed to suppress expression of the target gene (SEQ ID NO: 88) in Mon-DKD2.

### DETAILED DESCRIPTION

The present invention includes compositions and methods for producing haploid inducer plants, haploids, and doubled haploids (including the spontaneous diploidization). The definitions and methods provided herein define the present invention and guide those of ordinary skill in the art in the practice of the present invention. Without being bound by any theory, compositions and methods of the present invention may operate to achieve and enhance haploid induction effect in plants by genetically modifying the candidate genes and genetic elements disclosed herein.

As used herein, a “haploid” cell or nucleus comprises a single set of unpaired chromosomes (x). In contrast, a “diploid” cell or nucleus comprises two complete sets of chromosomes (2x) that are capable of homologous pairing. The haploid number of chromosomes can be represented by “n,” and the diploid number of chromosomes can be represented by “2n.” For example, in a diploid species such as corn, n=x=10, and 2n=2x=20. A polyploid cell or nucleus comprises more than two complete sets of chromosomes. For example, some wheat lines are hexaploids, meaning they contain three sets of paired chromosomes (2n=6x=42). Both diploid and polyploid cells and nuclei can be reduced to haploid states.

As used herein, a plant referred to as “doubled haploid” is developed by doubling the haploid set of chromosomes. In one aspect, a haploid plant provided herein undergoes spontaneous chromosome doubling. Spontaneous chromosome doubling can produce diploid sectors that give rise to normal diploid floral structures. Such spontaneously doubled sectors are desirable because diploid floral structures resulting from spontaneous chromosome doubling produce normal eggs and pollen that can be self-pollinated or used to perform crosses with other plants. A plant or seed that is obtained from a doubled haploid plant that is selfed to any number of generations may still be identified as a doubled haploid plant. A doubled haploid plant is considered a homozygous plant. A plant is considered to be doubled haploid if it is fertile, even if the entire vegetative part of the plant does not consist of the cells with the doubled set of chromosomes; that is, a plant will be considered doubled haploid if it contains viable gametes, even if it is chimeric.

The present invention provides methods facilitating the production of doubled haploid plants, which entails production of haploids followed by chromosome doubling. First a haploid inducer line is produced using the compositions and methods provided in the present invention. Then one or more lines are crossed with an inducer parent to produce haploids. Selection of haploids can be accomplished by various screening methods based on phenotypic or genotypic characteristics. In one approach, seeds resulting from a cross with an inducer parent are screened with visible marker genes, including anthocyanin genes such as R-nj and fluorescent proteins such as GFP YFP, CFP, DS-Red or CRC, that are detectable in the embryo of a haploid seed, allowing for separation of haploid and diploid seeds. The diploid seeds will contain the marker gene from the haploid inducer parent. Other screening approaches may be applied to plants resulting from the cross with the haploid inducer including chromosome counting, flow cytometry, and genetic marker evaluation can be utilized to infer genome copy number, etc. See U.S. Patent Application Publication No. 2009/0064361, the entire contents and disclosures of which are incorporated herein by reference.

The resulting haploid has a haploid embryo and a normal triploid endosperm. There are several approaches known in the art to achieve chromosome doubling. Haploid cells, haploid embryos, haploid seeds, haploid seedlings, or haploid plants can be treated with a doubling agent. Non-limiting examples of known doubling agents include nitrousoxide gas, anti-microtubule herbicides, anti-microtubule agents, colchicine, pronamide, and mitotic inhibitors. See U.S. Patent Application Publication No. 2014/0298532, the entire contents and disclosures of which are incorporated herein by reference.

As used herein, a “locus” is a fixed position on a chromosome and may represent a single nucleotide, a few nucleotides or a large number of nucleotides in a genomic region.

As used herein, “marker” means a detectable characteristic that can be used to discriminate between organisms. Examples of such characteristics may include genetic markers, protein composition, protein levels, oil composition, oil levels, carbohydrate composition, carbohydrate levels, fatty acid composition, fatty acid levels, amino acid composition, amino acid levels, biopolymers, pharmaceuticals, starch composition, starch levels, fermentable starch, fermentation yield, fermentation efficiency, energy yield, secondary compounds, metabolites, morphological characteristics, and agronomic characteristics.

As used herein, “genetic marker” means polymorphic nucleic acid sequence or nucleic acid feature. A “polymorphism” is a variation among individuals in sequence, particularly in DNA sequence, or feature, such as a transcriptional profile or methylation pattern. Useful polymorphisms include single nucleotide polymorphisms (SNPs), insertions or deletions in DNA sequence (Indels), simple sequence repeats of DNA sequence (SSRs) a restriction fragment length polymorphism, a haplotype, and a tag SNP. A genetic marker, a gene, a DNA-derived sequence, a RNA-derived sequence, a promoter, a 5′ untranslated region of a gene, a 3′ untranslated region of a gene, micro RNA, siRNA, a QTL, a satellite marker, a transgene, mRNA, ds mRNA, a transcriptional profile, and a methylation pattern may comprise polymorphisms.

As used herein, “marker assay” means a method for detecting a polymorphism at a particular locus using a particular method, e.g. measurement of at least one phenotype (such as seed color, flower color, or other visually detectable trait), restriction fragment length polymorphism (RFLP), single base extension, electrophoresis, sequence alignment, allelic specific oligonucleotide hybridization (ASO), random amplified polymorphic DNA (RAPD), micro array-based technologies, and nucleic acid sequencing technologies, etc.

As used herein, Mon-DKD2 and Mon-IDR1 are Monsanto commercially released corn inbreds that are non-haploid inducing.

As used herein, KHI1 is a corn maternal haploid inducer line derived from a genetic stock Stock6 (See U.S. Patent Application Publication No. 2004/0210959, the entire contents and disclosures of which are incorporated herein by reference). In addition to a high rate of maternal haploid induction, KHI1 also conditions strong anthocyanin pigmentation in the aleurone tissue in the crown region of the kernel and in the embryo. This visible marker can be used to identify the maternal haploids. The maternal haploid kernels possess colored crowns due to normal fertilization and development of the endosperm, but colorless embryos, if the female parent is non-pigmented (Birchler, 1994. In: Maize Handbook, Freeling & Walbot (eds) pp. 386-388; Chang, 1992. Maize Genetics Newsletter, 66:163-164).

As used herein, the term “crossed” or “cross” means the fusion of gametes via pollination to produce progeny (e.g., cells, seeds or plants). The term encompasses both sexual crosses (the pollination of one plant by another) and selfing (self-pollination, e.g., when the pollen and ovule are from the same plant).

As used herein, a “genetic map” is a description of genetic linkage relationships among loci on one or more chromosomes (or linkage groups) within a given species, generally depicted in a diagrammatic or tabular form. “Genetic mapping” is the process of defining the linkage relationships of loci through the use of genetic markers, populations segregating for the markers, and standard genetic principles of recombination frequency. A “genetic map location” is a location on a genetic map relative to surrounding genetic markers on the same linkage group where a specified marker can be found within a given species. If two different markers have the same genetic map location, the two markers are in such close proximity to each other that recombination occurs between them with such low frequency that it is undetectable.

As used herein, a “phenotypic marker” refers to a marker that can be used to discriminate phenotypes displayed by organisms.

As used herein, the term “transgene” means nucleic acid molecules in form of DNA, such as cDNA or genomic DNA, and RNA, such as mRNA or micro RNA, which may be single or double stranded.

The term “suppression” as used herein refers to a lower expression level of a target polynucleotide or target protein in a plant, plant cell or plant tissue, as compared to the expression in a wild-type or control plant, cell or tissue, at any developmental or temporal stage for the gene. The term “target protein” as used in the context of suppression refers to a protein which is suppressed; similarly, “target mRNA” refers to a polynucleotide which can be suppressed or, once expressed, degraded so as to result in suppression of the target protein it encodes. The term “target gene” as used in the context of suppression refers to either “target protein” or “target mRNA”. In alternate non-limiting embodiments, suppression of the target protein or target polynucleotide can give rise to an enhanced trait or altered phenotype directly or indirectly. In one exemplary embodiment, the target protein is one which can indirectly increase or decrease the expression of one or more other proteins, the increased or decreased expression, respectively, of which is associated with an enhanced trait or an altered phenotype. In another exemplary embodiment, the target protein can bind to one or more other proteins associated with an altered phenotype or enhanced trait to enhance or inhibit their function and thereby affect the altered phenotype or enhanced trait indirectly.

Suppression can be applied using numerous approaches. Non-limiting examples include: suppressing an endogenous gene(s) or a subset of genes in a pathway, suppressing one or more mutation that has resulted in decreased activity of a protein, suppressing the production of an inhibitory agent, to elevate, reduce or eliminate the level of substrate that an enzyme requires for activity, producing a new protein, activating a normally silent gene; or accumulating a product that does not normally increase under natural conditions.

The term “overexpression” as used herein refers to a greater expression level of a polynucleotide or a protein in a plant, plant cell or plant tissue, compared to expression in a wild-type plant, cell or tissue, at any developmental or temporal stage for the gene. Overexpression can take place in plant cells normally lacking expression of polypeptides functionally equivalent or identical to the present polypeptides. Overexpression can also occur in plant cells where endogenous expression of the present polypeptides or functionally equivalent molecules normally occurs, but such normal expression is at a lower level. Overexpression thus results in a greater than normal production, or “overproduction” of the polypeptide in the plant, cell or tissue.

Overexpression can be achieved using numerous approaches. In one embodiment, overexpression can be achieved by placing the DNA sequence encoding one or more polynucleotides or polypeptides under the control of a promoter, examples of which include but are not limited to endogenous promoters, heterologous promoters, inducible promoters and tissue specific promoters. In one exemplary embodiment, the promoter is a constitutive promoter, for example, the cauliflower mosaic virus 35S transcription initiation region. Thus, depending on the promoter used, overexpression can occur throughout a plant, in specific tissues of the plant, or in the presence or absence of different inducing or inducible agents, such as hormones or environmental signals.

The term “target protein” as used herein in the context of overexpression refers to a protein which is overexpressed; “target mRNA” refers to an mRNA which encodes and is translated to produce the target protein, which can also be overexpressed. The term “target gene” as used in the context of overexpression refers to either “target protein” or “target mRNA”. In alternative embodiments, the target protein can affect an enhanced trait or altered phenotype directly or indirectly. In the latter case it may do so, for example, by affecting the expression, function or substrate available to one or more other proteins. In an exemplary embodiment, the target protein can bind to one or more other proteins associated with an altered phenotype or enhanced trait to enhance or inhibit their function.

Gene Suppression Elements: The gene suppression element can be transcribable DNA of any suitable length, and generally includes at least about 19 to about 27 nucleotides (for example 19, 20, 21, 22, 23, or 24 nucleotides) for every target gene that the recombinant DNA construct is intended to suppress. In many embodiments the gene suppression element includes more than 23 nucleotides (for example, more than about 30, about 50, about 100, about 200, about 300, about 500, about 1000, about 1500, about 2000, about 3000, about 4000, or about 5000 nucleotides) for every target gene that the recombinant DNA construct is intended to suppress.

Suitable gene suppression elements useful in the recombinant DNA constructs of the invention include at least one element (and, in some embodiments, multiple elements) selected from the group consisting of: (a) DNA that includes at least one anti-sense DNA segment that is anti-sense to at least one segment of the at least one first target gene; (b) DNA that includes multiple copies of at least one anti-sense DNA segment that is anti-sense to at least one segment of the at least one first target gene; (c) DNA that includes at least one sense DNA segment that is at least one segment of the at least one first target gene; (d) DNA that includes multiple copies of at least one sense DNA segment that is at least one segment of the at least one first target gene; (e) DNA that transcribes to RNA for suppressing the at least one first target gene by forming double-stranded RNA and includes at least one anti-sense DNA segment that is anti-sense to at least one segment of the at least one target gene and at least one sense DNA segment that is at least one segment of the at least one first target gene; (f) DNA that transcribes to RNA for suppressing the at least one first target gene by forming a single double-stranded RNA and includes multiple serial anti-sense DNA segments that are anti-sense to at least one segment of the at least one first target gene and multiple serial sense DNA segments that are at least one segment of the at least one first target gene; (g) DNA that transcribes to RNA for suppressing the at least one first target gene by forming multiple double strands of RNA and includes multiple anti-sense DNA segments that are anti-sense to at least one segment of the at least one first target gene and multiple sense DNA segments that are at least one segment of the at least one first target gene, and wherein said multiple anti-sense DNA segments and the multiple sense DNA segments are arranged in a series of inverted repeats; (h) DNA that includes nucleotides derived from a miRNA, preferably a plant miRNA; (i) DNA that includes nucleotides of a siRNA; (j) DNA that transcribes to an RNA aptamer capable of binding to a ligand; and (k) DNA that transcribes to an RNA aptamer capable of binding to a ligand, and DNA that transcribes to regulatory RNA capable of regulating expression of the first target gene, wherein the regulation is dependent on the conformation of the regulatory RNA, and the conformation of the regulatory RNA is allosterically affected by the binding state of the RNA aptamer.

As used herein a “plant” includes a whole plant, a transgenic plant, meristematic tissue, a shoot organ/structure (for example, leaf, stem and tuber), a root, a flower, a floral organ/structure (for example, a bract, a sepal, a petal, a stamen, a carpel, an anther and an ovule), a seed (including an embryo, endosperm, and a seed coat) and a fruit (the mature ovary), plant tissue (for example, vascular tissue, ground tissue, and the like) and a cell (for example, guard cell, egg cell, pollen, mesophyll cell, and the like), and progeny of same. The classes of plants that can be used in the disclosed methods are generally as broad as the classes of higher and lower plants amenable to transformation and breeding techniques, including angiosperms (monocotyledonous and dicotyledonous plants), gymnosperms, ferns, horsetails, psilophytes, lycophytes, bryophytes, and multicellular algae.

As used herein a “transgenic plant cell” means a plant cell that is transformed with stably-integrated, recombinant DNA, for example, by Agrobacterium-mediated transformation or by bombardment using microparticles coated with recombinant DNA or by other means. A plant cell of this disclosure can be an originally-transformed plant cell that exists as a microorganism or as a progeny plant cell that is regenerated into differentiated tissue, for example, into a transgenic plant with stably-integrated, recombinant DNA, or seed or pollen derived from a progeny transgenic plant.

As used herein a “recombinant polynucleotide” or “recombinant DNA” is a polynucleotide that is not in its native state, for example, a polynucleotide comprises a series of nucleotides (represented as a nucleotide sequence) not found in nature, or a polynucleotide is in a context other than that in which it is naturally found; for example, separated from polynucleotides with which it typically is in proximity in nature, or adjacent (or contiguous with) polynucleotides with which it typically is not in proximity The “recombinant polynucleotide” or “recombinant DNA” refers to polynucleotide or DNA which has been genetically engineered and constructed outside of a cell including DNA containing naturally occurring DNA or cDNA or synthetic DNA. For example, the polynucleotide at issue can be cloned into a vector, or otherwise recombined with one or more additional nucleic acids.

As used herein, a “functional fragment” refers to a portion of a polypeptide provided herein which retains full or partial molecular, physiological or biochemical function of the full length polypeptide. In the present invention, transformation constructs can be made to contain portions of the causal genetic elements associated with haploid induction. Each of these constructs may be transformed into a non-haploid induction line and the resulting transgenic plants are evaluated for haploid induction. By testing several such constructs that contain different fragments of the causal genetic elements, the portion required for haploid induction can determined.

A “recombinant DNA construct” as used in the present disclosure comprises at least one expression cassette having a promoter operable in plant cells and a polynucleotide of the present disclosure. DNA constructs can be used as a means of delivering recombinant DNA constructs to a plant cell in order to effect stable integration of the recombinant molecule into the plant cell genome. In one embodiment, the polynucleotide can encode a protein or variant of a protein or fragment of a protein that is functionally defined to maintain activity in transgenic host cells including plant cells, plant parts, explants and whole plants. In another embodiment, the polynucleotide can encode a non-coding RNA that interferes with the functioning of endogenous classes of small RNAs that regulate expression, including but not limited to taRNAs, siRNAs and miRNAs. Recombinant DNA constructs are assembled using methods known to persons of ordinary skill in the art and typically comprise a promoter operably linked to DNA, the expression of which provides the enhanced agronomic trait.

Percent identity describes the extent to which polynucleotides or protein segments are invariant in an alignment of sequences, for example nucleotide sequences or amino acid sequences. An alignment of sequences is created by manually aligning two sequences, for example, a stated sequence, as provided herein, as a reference, and another sequence, to produce the highest number of matching elements, for example, individual nucleotides or amino acids, while allowing for the introduction of gaps into either sequence. An “identity fraction” for a sequence aligned with a reference sequence is the number of matching elements, divided by the full length of the reference sequence, not including gaps introduced by the alignment process into the reference sequence. “Percent identity” (“% identity”) as used herein is the identity fraction times 100.

As used herein, a “homolog” or “homologues” means a protein in a group of proteins that perform the same biological function, for example, proteins that belong to the same Pfam protein family and that provide a common enhanced trait in transgenic plants of this disclosure. Homologs are expressed by homologous genes. With reference to homologous genes, homologs include orthologs, for example, genes expressed in different species that evolved from common ancestral genes by speciation and encode proteins retain the same function, but do not include paralogs, i.e., genes that are related by duplication but have evolved to encode proteins with different functions. Homologous genes include naturally occurring alleles and artificially-created variants.

As used herein, the term “promoter” refers generally to a DNA molecule that is involved in recognition and binding of RNA polymerase II and other proteins (trans-acting transcription factors) to initiate transcription. A promoter can be initially isolated from the 5′ untranslated region (5′ UTR) of a genomic copy of a gene. Alternately, promoters can be synthetically produced or manipulated DNA molecules. Promoters can also be chimeric, that is a promoter produced through the fusion of two or more heterologous DNA molecules. Plant promoters include promoter DNA obtained from plants, plant viruses, fungi and bacteria such as Agrobacterium and Bradyrhizobium bacteria.

Promoters which initiate transcription in all or most tissues of the plant are referred to as “constitutive” promoters. Promoters which initiate transcription during certain periods or stages of development are referred to as “developmental” promoters. Promoters whose expression is enhanced in certain tissues of the plant relative to other plant tissues are referred to as “tissue enhanced” or “tissue preferred” promoters. Promoters which express within a specific tissue of the plant, with little or no expression in other plant tissues are referred to as “tissue specific” promoters. A promoter that expresses in a certain cell type of the plant, for example a microspore mother cell, is referred to as a “cell type specific” promoter. An “inducible” promoter is a promoter in which transcription is initiated in response to an environmental stimulus such as cold, drought or light; or other stimuli such as wounding or chemical application. Many physiological and biochemical processes in plants exhibit endogenous rhythms with a period of about 24 hours. A “diurnal promoter” is a promoter which exhibits altered expression profiles under the control of a circadian oscillator. Diurnal regulation is subject to environmental inputs such as light and temperature and coordination by the circadian clock.

Sufficient expression in plant seed tissues is desired to affect improvements in seed composition. Exemplary promoters for use for seed composition modification include promoters from seed genes such as napin as disclosed in U.S. Pat. No. 5,420,034, maize L3 oleosin as disclosed in U.S. Pat. No. 6,433,252, zein Z27 as disclosed by Russell et al. (1997) Transgenic Res. 6(2):157-166, globulin 1 as disclosed by Belanger et al (1991) Genetics 129:863-872, glutelin 1 as disclosed by Russell (1997) supra, and peroxiredoxin antioxidant (Per1) as disclosed by Stacy et al. (1996) Plant Mol Biol. 31(6):1205-1216.

Expression cassettes of this disclosure can include a “transit peptide” or “targeting peptide” or “signal peptide” molecule located either 5′ or 3′ to or within the gene(s). These terms generally refer to peptide molecules that when linked to a protein of interest directs the protein to a particular tissue, cell, subcellular location, or cell organelle. Examples include, but are not limited to, chloroplast transit peptides (CTPs), chloroplast targeting peptides, mitochondrial targeting peptides, nuclear targeting signals, nuclear exporting signals, vacuolar targeting peptides, and vacuolar sorting peptides. For description of the use of chloroplast transit peptides see U.S. Pat. Nos. 5,188,642 and 5,728,925. For description of the transit peptide region of an Arabidopsis EPSPS gene in the present disclosure, see Klee, H. J. Et al (MGG (1987) 210:437-442. Expression cassettes of this disclosure can also include an intron or introns. Expression cassettes of this disclosure can contain a DNA near the 3′ end of the cassette that acts as a signal to terminate transcription from a heterologous nucleic acid and that directs polyadenylation of the resultant mRNA. These are commonly referred to as “3′-untranslated regions” or “3′-non-coding sequences” or “3′-UTRs”. The “3′ non-translated sequences” means DNA sequences located downstream of a structural nucleotide sequence and include sequences encoding polyadenylation and other regulatory signals capable of affecting mRNA processing or gene expression. The polyadenylation signal functions in plants to cause the addition of polyadenylate nucleotides to the 3′ end of the mRNA precursor. The polyadenylation signal can be derived from a natural gene, from a variety of plant genes, or from T-DNA. An example of a polyadenylation sequence is the nopaline synthase 3′ sequence (nos 3′; Fraley et al., Proc. Natl. Acad. Sci. USA 80: 4803-4807, 1983). The use of different 3′ non-translated sequences is exemplified by Ingelbrecht et al., Plant Cell 1:671-680, 1989.

Expression cassettes of this disclosure can also contain one or more genes that encode selectable markers and confer resistance to a selective agent such as an antibiotic or an herbicide. A number of selectable marker genes are known in the art and can be used in the present disclosure: selectable marker genes conferring tolerance to antibiotics like kanamycin and paromomycin (nptII), hygromycin B (aph IV), spectinomycin (aadA), U.S. Patent Publication 2009/0138985A1 and gentamycin (aac3 and aacC4) or tolerance to herbicides like glyphosate (for example, 5-enolpyruvylshikimate-3-phosphate synthase (EPSPS), U.S. Pat. Nos. 5,627,061; 5,633,435; 6,040,497; 5,094,945), sulfonyl herbicides (for example, acetohydroxyacid synthase or acetolactate synthase conferring tolerance to acetolactate synthase inhibitors such as sulfonylurea, imidazolinone, triazolopyrimidine, pyrimidyloxybenzoates and phthalide (U.S. Pat. Nos. 6,225,105; 5,767,366; 4,761,373; 5,633,437; 6,613,963; 5,013,659; 5,141,870; 5,378,824; 5,605,011)), bialaphos or phosphinothricin or derivatives (e. g., phosphinothricin acetyltransferase (bar) tolerance to phosphinothricin or glufosinate (U.S. Pat. Nos. 5,646,024; 5,561,236; 5,276,268; 5,637,489; 5,273,894); dicamba (dicamba monooxygenase, Patent Application Publications US2003/0115626A1), or sethoxydim (modified acetyl-coenzyme A carboxylase for conferring tolerance to cyclohexanedione), and aryloxyphenoxypropionate (haloxyfop, U.S. Pat. No. 6,414,222).

Transformation vectors of this disclosure can contain one or more “expression cassettes”, each comprising a native or non-native plant promoter operably linked to a polynucleotide sequence of interest, which is operably linked to a 3′ UTR sequence and termination signal, for expression in an appropriate host cell. It also typically comprises sequences required for proper translation of the polynucleotide or transgene. As used herein, the term “transgene” refers to a polynucleotide molecule artificially incorporated into a host cell's genome. Such a transgene can be heterologous to the host cell. The term “transgenic plant” refers to a plant comprising such a transgene. The coding region usually codes for a protein of interest but can also code for a functional RNA of interest, for example an antisense RNA, a nontranslated RNA, in the sense or antisense direction, a miRNA, a noncoding RNA, or a synthetic RNA used in either suppression or over expression of target gene sequences. The expression cassette comprising the nucleotide sequence of interest can be chimeric, meaning that at least one of its components is heterologous with respect to at least one of its other components. As used herein the term “chimeric” refers to a DNA molecule that is created from two or more genetically diverse sources, for example a first molecule from one gene or organism and a second molecule from another gene or organism.

Recombinant DNA constructs in this disclosure generally include a 3′ element that typically contains a polyadenylation signal and site. Known 3′ elements include those from Agrobacterium tumefaciens genes such as nos 3′, tml 3′, tmr 3′, tms 3′, ocs 3′, tr7 3′, for example disclosed in U.S. Pat. No. 6,090,627; 3′ elements from plant genes such as wheat (Triticum aesevitum) heat shock protein 17 (Hsp17 3′), a wheat ubiquitin gene, a wheat fructose-1,6-biphosphatase gene, a rice glutelin gene, a rice lactate dehydrogenase gene and a rice beta-tubulin gene, all of which are disclosed in U.S. Patent Application Publication 2002/0192813 A1; and the pea (Pisum sativum) ribulose biphosphate carboxylase gene (rbs 3′), and 3′ elements from the genes within the host plant.

As used herein “operably linked” means the association of two or more DNA fragments in a recombinant DNA construct so that the function of one, for example, protein-encoding DNA, is controlled by the other, for example, a promoter.

Transgenic plants can comprise a stack of one or more polynucleotides disclosed herein resulting in the production of multiple polypeptide sequences. Transgenic plants comprising stacks of polynucleotides can be obtained by either or both of traditional breeding methods or through genetic engineering methods. These methods include, but are not limited to, crossing individual transgenic lines each comprising a polynucleotide of interest, transforming a transgenic plant comprising a first gene disclosed herein with a second gene, and co-transformation of genes into a single plant cell. Co-transformation of genes can be carried out using single transformation vectors comprising multiple genes or genes carried separately on multiple vectors.

Transgenic plants comprising or derived from plant cells of this disclosure transformed with recombinant DNA can be further enhanced with stacked traits, for example, a crop plant having an enhanced trait resulting from expression of DNA disclosed herein in combination with herbicide and/or pest resistance traits. For example, genes of the current disclosure can be stacked with other traits of agronomic interest, such as a trait providing herbicide resistance, or insect resistance, such as using a gene from Bacillus thuringensis to provide resistance against lepidopteran, coliopteran, homopteran, hemiopteran, and other insects, or improved quality traits such as improved nutritional value. Herbicides for which transgenic plant tolerance has been demonstrated and the method of the present disclosure can be applied include, but are not limited to, glyphosate, dicamba, glufosinate, sulfonylurea, bromoxynil, norflurazon, 2,4-D (2,4-dichlorophenoxy)acetic acid, aryloxyphenoxy propionates, p-hydroxyphenyl pyruvate dioxygenase inhibitors (HPPD), and protoporphyrinogen oxidase inhibitors (PPO) herbicides. Polynucleotide molecules encoding proteins involved in herbicide tolerance known in the art and include, but are not limited to, a polynucleotide molecule encoding 5-enolpyruvylshikimate-3-phosphate synthase (EPSPS) disclosed in U.S. Pat. Nos. 5,094,945; 5,627,061; 5,633,435 and 6,040,497 for imparting glyphosate tolerance; polynucleotide molecules encoding a glyphosate oxidoreductase (GOX) disclosed in U.S. Pat. No. 5,463,175 and a glyphosate-N-acetyl transferase (GAT) disclosed in U.S. Patent No. Application Publication 2003/0083480 A1 also for imparting glyphosate tolerance; dicamba monooxygenase disclosed in U.S. Patent Application Publication 2003/0135879 A1 for imparting dicamba tolerance; a polynucleotide molecule encoding bromoxynil nitrilase (Bxn) disclosed in U.S. Pat. No. 4,810,648 for imparting bromoxynil tolerance; a polynucleotide molecule encoding phytoene desaturase (crtI) described in Misawa et al, (1993) Plant J. 4:833-840 and in Misawa et al, (1994) Plant J. 6:481-489 for norflurazon tolerance; a polynucleotide molecule encoding acetohydroxyacid synthase (AHAS, aka ALS) described in Sathasiivan et al. (1990) Nucl. Acids Res. 18:2188-2193 for imparting tolerance to sulfonylurea herbicides; polynucleotide molecules known as bar genes disclosed in DeBlock, et al. (1987) EMBO J. 6:2513-2519 for imparting glufosinate and bialaphos tolerance; polynucleotide molecules disclosed in U.S. Patent Application Publication 2003/010609 A1 for imparting N-amino methyl phosphonic acid tolerance; polynucleotide molecules disclosed in U.S. Pat. No. 6,107,549 for imparting pyridine herbicide resistance; molecules and methods for imparting tolerance to multiple herbicides such as glyphosate, atrazine, ALS inhibitors, isoxoflutole and glufosinate herbicides are disclosed in U.S. Pat. No. 6,376,754 and U.S. Patent Application Publication 2002/0112260. Molecules and methods for imparting insect/nematode/virus resistance are disclosed in U.S. Pat. Nos. 5,250,515; 5,880,275; 6,506,599; 5,986,175 and U.S. Patent Application Publication 2003/0150017 A1.

As an alternative to traditional transformation methods, a DNA sequence, such as a transgene, expression cassette(s), etc., may be inserted or integrated into a specific site or locus within the genome of a plant or plant cell via site-directed integration. Recombinant DNA construct(s) and molecule(s) of this disclosure may thus include a donor template sequence comprising at least one transgene, expression cassette, or other DNA sequence for insertion into the genome of the plant or plant cell. Such donor template for site-directed integration may further include one or two homology arms flanking the sequence, transgene, cassette, etc., to be inserted into the plant genome. The recombinant DNA construct(s) of this disclosure may further comprise an expression cassette(s) encoding a site-specific nuclease and/or any associated protein(s) to carry out site-directed integration. These nuclease expressing cassette(s) may be present in the same molecule or vector as the donor template (in cis) or on a separate molecule or vector (in trans). Several methods for site-directed integration are known in the art involving different proteins (or complexes of proteins and/or guide RNA) that cut the genomic DNA to produce a double strand break (DSB) or nick at a desired genomic site or locus. Briefly as understood in the art, during the process of repairing the DSB or nick introduce by the nuclease enzyme, the donor template DNA may become integrated into the genome at the site of the DSB or nick. The presence of the homology arm(s) in the donor template may promote the adoption and targeting of the insertion sequence into the plant genome during the repair process through homologous recombination, although an insertion event may occur through non-homologous end joining (NHEJ). Examples of site-specific nucleases that may be used include zinc-finger nucleases, engineered or native meganucleases, TALE-endonucleases, and RNA-guided endonucleases (e.g., Cas9 or Cpf1). For methods using RNA-guided site-specific nucleases (e.g., Cas9 or Cpf1), the recombinant DNA construct(s) will also comprise a sequence encoding one or more guide RNAs to direct the nuclease to the desired site within the plant genome.

As used herein, the term “homology arm” refers to a polynucleotide sequence that has at least 80%, at least 85%, at least 90%, at least 91%, at least 92%, at least 93%, at least 94%, at least 95%, at least 96%, at least 97%, at least 98%, at least 99% or 100% sequence identity to a target sequence in a plant or plant cell that is being transformed. A homology arm can comprise at least 15, at least 20, at least 25, at least 30, at least 40, at least 50, at least 100, at least 250, at least 500, or at least 1000 nucleotides.

In the present invention, genomic region corresponding to the haploid induction region MonI1 in non-haploid inducer lines can be edited to create haploid inducer lines. Custom nucleases may be designed to cut sequences flanking the region from a non-haploid induction line that corresponds to the haploid induction region. The nucleases may be TALENs, ZFNs, CRISPR/Cas9, meganucleases, or other custom nucleases. As an alternative to custom nucleases, custom recombinases may be designed to target sequences flanking the region from a non-haploid inducer line that corresponds to the haploid induction region. A WT line such as corn Mon-DKD2 is transformed with constructs expressing the nucleases or recombinases and events screened to identify cases where the region was removed.

Progeny of those cases may be screened for homozygous deletions and then crossed to a tester with polymorphic genetic markers. The progeny are harvested and scored for haploids.

To determine which region, when lost, is responsible for haploid induction, additional nucleases or recombinases may be designed and used to delete portions of the region corresponding to haploid induction locus. Events may be produced using these reagents and screened to find the intended deletions. Progeny may be screened for homozygous deletions and then crossed to a tester with polymorphic genetic markers. The progeny are harvested and scored for haploids.

As used herein, the term “supernumerary chromosome” refers to an extra chromosome found in addition to the normal complement of A chromosomes. In one aspect, a HI inducer line provided herein comprises at least 1, 2, 3, 4, 5, 6, 7, 8, 9, or at least 10 supernumerary chromosomes. In another aspect, a HI non-inducer line provided herein comprises at least 1, 2, 3, 4, 5, 6, 7, 8, 9, or at least 10 supernumerary chromosomes. In one aspect, a supernumerary chromosome provided herein is a B chromosome. In another aspect, a supernumerary chromosome provided herein is an artificially derived chromosome. In yet another aspect, an artificially derived chromosome provided herein is a truncated chromosome or a de novo generated chromosome.

In an aspect, a B chromosome provided herein is a maize B chromosome. In another aspect, a B chromosome provided herein is a rye B chromosome. In an aspect, a B chromosome provided herein is a Tripsacum B chromosome. B chromosomes are found in addition to the normal diploid complement of chromosomes in a cell. For example, in maize, the normal diploid complement of chromosomes is 20. B chromosomes are dispensable and are not required for normal plant development. When two B chromosomes are present in a single plant, the two B chromosomes can pair with each other at meiotic prophase and recombination can occur. B chromosomes do not pair with or recombine with A chromosomes.

In one aspect, a method provided herein comprises the incorporation of a DNA of interest into a supernumerary chromosome. In another aspect, a method provided herein comprises the modification at least one locus on a supernumerary chromosome. In another aspect, a method provided herein comprises the translocation of a nucleic acid molecule from a supernumerary chromosome to an A chromosome, a plastid genome, or a mitochondrial genome.

One or more B chromosomes, according to certain aspects of the present disclosure, can be delivered to a progeny plant without the rest of the paternal or maternal genome (e.g., via a haploid induction cross that retains the B chromosome), allowing complete conversion to a new variety in a single cross. In another aspect, a B chromosome may be transferred from a first plant species to a second plant species, allowing testing of the transgene or transgenes in other crops. For example, transmission of a B chromosome to oat has been demonstrated, as well as transmission of a corn chromosome to wheat (Koo et al., Genome Research 21(6):908-914, 2011; Comeau et al., Plant Science 81(1):117-125, 1992).

In certain cases, such as in corn and rye, B chromosomes have “accumulation mechanisms” that allow them to transmit at greater than Mendelian frequencies. For example, in corn, the sister chromatids of the B chromosome fail to separate during the second pollen (first generative) division. As a result, both sister chromatids are delivered to one of the sperm, while the other receives neither. This effect, called non-disjunction, means that a plant with only a single B chromosome can deliver zero, one, or two B chromosomes to the next generation when used as a male. Such an effect may be desirable during the trait introgression process, since it allows individuals that are homozygous (as opposed to hemizygous) for a megalocus carried on a B chromosome to be recovered in a backcross, as long as the B chromosome is delivered from the pollen.

In another aspect, B chromosomes may be used to rapidly transfer the haploid induction effect to new lines. Because the B chromosome can be retained at a low percentage, a line where the haploid induction effect is caused by the B chromosome may allow the effect to be moved to additional lines by a single cross. This simplifies creation of new haploid induction lines with desired agronomic or genetic properties. Genetic elements disclosed in this application can be incorporated into a B chromosome to produce plants containing the haploid inducing B chromosome (HI-B chromosome). Other haploid induction genes, e.g. the CENH3-based transgenes (Kelliher, T et al., “Maternal Haploids Are Preferentially Induced by CENH3-tailswap Transgenic Complementation in Maize”, Frontiers in Plant Science 7: 414 (2016)) can also be incorporated into a B chromosome to produce plants containing HI-B chromosome. To move the haploid induction effect to new lines, a line containing HI-B chromosome is crossed to the desired line and progeny are screened for cases that are haploid and that have retained the HI-B chromosome.

In another embodiment, a non-inducer line can be converted to a haploid inducer line by either deleting the region corresponding to MonI1 in the non-inducer line, or swap the entire or portions of such region with the haploid induction region in KHI1.

### EXAMPLES

### Example 1. Identification of the Fine Mapped Haploid Induction Region (MonI1) and the Associated Causal Genetic Elements

In this example, the haploid induction QTL (qhir1) was fine mapped using backcross progenies of corn non-inducer line Mon-IDR1 and inducer line KHI1, and the Genotyping by Sequencing (GBS) was used for high density mapping. 276 GBS markers from 8 MB region in 100-105 cM of the haploid induction QTL in Chromosome 1 were used in the fine mapping process. The haploid induction locus was narrowed down to a 238 kb MonI1 region (SEQ ID NO: 6) flanked by markers MonI1-m1 (SEQ ID NO: 7) and MonI1-m2 (SEQ ID NO: 8) based on the B73 reference genome. The SNP positions and genotypes of both markers are listed in Table 1.

TABLE 1
Genetic markers associated with haploid
induction for the MonI1 region.

| Marker | Map Position | SEQ ID | SNP | Allelic Homozygous |
|---|---|---|---|---|
| Name | in B73 (cM) | NO: | Position | forms (KHI1/Mon-IDR1) |
| MonI1-m1 | 104.67 | 44 | 101 | GG/TT |
| MonI1-m2 | 104.8 | 45 | 101 | GG/AA |

BAC library was also created for the haploid inducer line KHI1 and screened using similar set of GBS markers used in the fine mapping process. Eleven overlapping BACs spanning the extended region of the MonI1 were identified and sequenced using PacBio and Illumina. Assembly of the eleven BACs formed an about 400 kb contiguous sequence (SEQ ID NO: 1). Three KHI1 BAC sequences (SEQ ID NOs: 2, 3 and 4) were identified that spanned the MonI1 region and the contiguous sequence (SEQ ID NO: 5) flanked by the MonI1 markers was extracted from the assembly (SEQ ID NO: 1) to represent the MonI1 region in KHI1.

Sequence analysis of the KHI1 assembly (SEQ ID NO: 1) identified a number of DNA coding sequences (SEQ ID NOs: 9-30) and among them, 5 coding DNA sequences (SEQ ID NOs 20-24) were identified within the KHI1 MonI1 region (SEQ ID NO: 5). For example, SEQ ID NO: 20 is a coding DNA sequence encoding a patatin-like phospholipase (PNPLA, SEQ ID NO: 42). The PNPLA gene was identified in the MonI1 region in both B73 (PNPLA, SEQ ID NO: 91) and KHI1. The Homologous PNPLA genes were also identified in rice (SEQ ID NO: 107) and sorghum (SEQ ID NO: 109). More PNPLA homologs from other species such as wheat and brachypodium are also known in the public databases. 7 genes in B73 MonI1 region (SEQ ID NOs: 93 and 99) were identified to be absent in the KHI1 MonI1 region. Sequence analysis of the MonI1 region in corn Mon-DKD2 showed high homology to B73. For examples, the Mon-DKD2 genes (SEQ ID NOs: 87 and 88) are homologous to the B73 genes (SEQ ID NO: 94 and 95), respectively. A number of the ncRNAs (SEQ ID NOs: 53-85) were predicted within the assembly (SEQ ID NO: 1) in KHI1 and among these ncRNAs, SEQ ID NOs: 72-82 were identified within the MonI1 region (SEQ ID NO: 5) in KHI1.

The KHI1 MonI1 sequences and the shared, deleted or unique genetic elements identified above are candidates for producing transgenic haploid inducer plants.

### Example 2. Validation of Haploid Induction Phenotype in Corn Caused by the KHI1 MonI1 Sequence

One of the KHI1 MonI1 sequences represented by SEQ ID NOs: 1-5 and their complements or fragments is transformed into corn by Agrobacterium-Mediated transformation of dry excised embryo's from a non-haploid inducing maize line and selected using glyphosate. The resulting R0 transgenic plants are assayed with molecular markers from the haploid induction line that are present in the BAC but not present in the non-haploid inducing maize line. The markers are distributed throughout the BAC and several events are identified that contained all the markers showing that the entire BAC was present.

The R0 plants from these events and events that lacked some or all of the markers from the KHI1 BAC are self pollinated and seeds are harvested. These seeds are planted and seedlings are genotyped to identify individuals that are homozygous for the transgenic insertion. These individuals are grown and used as pollen sources to cross onto wild-type plants of a different variety (genetic markers are available to distinguish genotypes of the two parent lines). As a further precaution to ensure that self-pollination of the WT parent does not occur, tassels from all WT parents are removed before maturity.

Next, seeds are harvested from these crosses and germinated. The seedlings are screened by genotyping to identify cases that lack the transgene or genetic markers from the non-haploid inducing maize line. These cases represent putative haploids.

### Example 3. Use of the KHI1 MonI1 Sequence to Create Haploid Inducer Lines in Corn Relatives

The construct containing KHI1 MonI1 sequences identified in Example 1 is transformed into sorghum. The resulting transgenic events are screened by molecular methods to confirm construct intactness. R0 individuals are self pollinated and R1 progeny are screened for homozygous individuals. These individuals are crossed to WT sorghum of a different variety. The progeny of this haploid induction test cross are assayed with genetic markers that detect the transgene and/or the transformation line. Cases where no markers from the transgenic parent are detected are candidate haploids.

### Example 4. Over-Expression of MonI1 Candidate Gene to Produce Haploid Induction Lines

A construct comprising the polynucleotide sequence selected from the group consisting of SEQ ID NOs: 20-24, and 86 is created. This construct is transformed into a WT non-inducer line (Mon-DKD2) using agrobacteria and events are selected that had an intact single copy T-DNA. The events are self-pollinated to produce R1 seed.

R1 plants are germinated and sampled. Individuals that are homozygous (2 copy) for the events are selected and grown. These plants are crossed to a different non-haploid induction line and progeny are evaluated with markers to select haploid lines as described in Example 2.

### Example 5. Over-Expression of MonI1 Candidate Gene Stack to Produce Haploid Inducer Lines

A construct comprising a first polynucleotide sequence and a second polynucleotide sequence selected from the group consisting of SEQ ID NOs: 20-24 is created. This construct is transformed into a WT non-inducer line (Mon-DKD2) using agrobacteria and events are selected that have an intact single copy T-DNA. The events are self-pollinated to produce R1 seed.

R1 plants are germinated and sampled. Individuals that are homozygous (2 copy) for the events are selected and grown. These plants are crossed to a different non-haploid induction line and progeny are evaluated with markers to select haploid lines as described in Example 2.

### Example 6. Over-Expression of Individual Candidate Gene or Gene Stack to Cause Haploid Induction in Sorghum

The constructs described in Example 4 or 5 are transformed into sorghum. The resulting transgenic events are screened by molecular methods to confirm construct intactness. R0 individuals are self pollinated and R1 progeny are screened for homozygous individuals. These individuals are crossed to WT sorghum of a different variety. The progeny of this haploid induction test cross are assayed with genetic markers that detect the transgene and/or the transformation line. Cases where no markers from the transgenic parent are detected are candidate haploids.

### Example 7. Suppression of Candidate Target Genes to Cause Haploid Induction

A construct was made to express an artificial miRNA (SEQ ID NO: 111) to suppress expression of the target gene (SEQ ID NO: 87) identified in Mon-DKD2 in Example 1. This construct was transformed into a non-inducer line using agrobacteria and events were selected that had an intact single copy T-DNA. The events were self-pollinated and seed harvested.

R1 plants from several events were germinated and sampled. Individuals that were homozygous (2 copy) for the events were selected and are grown. These plants are crossed to a different inbred line and progeny evaluated to determine if any are haploid as described in Example 2.

Another construct was also made to express an artificial miRNA (SEQ ID NO: 112) to suppress expression of the target gene (SEQ ID NO: 88). This construct is tested and determined for haploid induction effect as described above.

A construct is made to express a suppression element to suppress expression of an endogenous target gene that is present in the MonI1 region in the target species but absent in the KHI MonI region. This construct is tested and determined for haploid induction effect as described above.

### Example 8. Expression of Non-Coding RNAs to Cause Haploid Induction

Constructs are made that have expression cassettes for one or more of the ncRNAs (SEQ ID NOs: 72-82) present in the KHI MonI1 region. Events are made and tested for haploid induction as described in previous examples.

1. A recombinant DNA construct comprising a promoter functional in a plant cell and operably linked to:
a) a polynucleotide that comprises a nucleotide sequence with at least 90%, at least 91%, at least 92%, at least 93%, at least 94%, at least 95%, at least 96%, at least 97%, at least 98%, at least 99% identity, or 100% identity to a sequence selected from the group consisting of SEQ ID NOs: 1-5, and their complements, or a functional fragment thereof;
b) a polynucleotide that comprises a nucleotide sequence with at least 90%, at least 91%, at least 92%, at least 93%, at least 94%, at least 95%, at least 96%, at least 97%, at least 98%, at least 99% identity, or 100% identity to a sequence selected from the group consisting of SEQ ID NOs: 20-24, 86, 107 and 109;
c) a polynucleotide that encodes a polypeptide having an amino acid sequence with at least 90%, at least 91%, at least 92%, at least 93%, at least 94%, at least 95%, at least 96%, at least 97%, at least 98%, at least 99% identity, or 100% identity to a sequence selected from the group consisting of SEQ ID NOs: 42-46, 108 and 110; or
d) a polynucleotide that comprises a nucleotide sequence suppressing at least one endogenous target gene having an amino acid sequence with at least 90%, at least 91%, at least 92%, at least 93%, at least 94%, at least 95%, at least 96%, at least 97%, at least 98%, at least 99% identity, or 100% identity to a sequence selected from the group consisting of SEQ ID NOs: 89, 90, 92 and 100-106.

2. The recombinant DNA construct of claim 1, wherein said nucleotide sequence suppressing at least one endogenous target gene is selected from the group consisting of SEQ ID NOs: 111 and 112.

3. A DNA molecule or vector comprising the recombinant DNA construct of claim 1.

4. A transgenic plant comprising the recombinant DNA construct of claim 1.

5. The transgenic plant of claim 4, wherein said plant is a progeny, a propagule, or a field crop.

6. The transgenic plant of claim 4, wherein said plant is a propagule selected from the group consisting of cell, pollen, ovule, flower, embryo, leaf, root, stem, shoot, meristem, grain and seed.

7. The transgenic plant of claim 4, wherein said plant is a field crop selected from the group consisting of corn, soybean, sorghum, cotton, canola, rice, barley, oat, wheat, turf grass, alfalfa, sugar beet, sunflower, quinoa and sugar cane.

8. (canceled)

9. (canceled)

10. (canceled)

11. (canceled)

12. (canceled)

13. A method for obtaining a haploid inducer plant comprising the steps of:
a) transforming at least one cell of an explant with a recombinant DNA construct of claim 1;
b) regenerating or developing the transgenic plant from the transformed explants; and
c) selecting a plant that exhibits haploid induction phenotype when crossed to a non-inducer line.

14. The method of claim 8, wherein the transforming step (a) is carried out via Agrobacterium-mediated transformation or microprojectile bombardment of the explant.

15. The method of claim 8, wherein the transforming step (a) comprises site-directed integration of the recombinant DNA construct.

16. A method for obtaining a haploid inducer plant comprising the steps of:
a) identifying an endogenous genomic locus corresponding to a gene selected from the group consisting of SEQ ID NOs: 42-46, 89-90, 92, 100-106, 108 and 110, or its homologs; and
b) site-specifically inserting a recombinant sequence capable of modulating expression of said gene by transforming the plant with a recombinant DNA construct

17. The method of claim 11, wherein said recombinant DNA construct comprises a donor template, wherein said donor template comprises at least one homology arm flanking a recombinant sequence for modulation of expression of an endogenous gene.

18. The method of claim 11, wherein said recombinant DNA construct further comprises at least one cassette encoding site-specific nuclease, wherein said site specific nuclease is selected from the group comprising zinc-finger nuclease, an engineered or native meganuclease, a TALE-endonuclease, or an RNA-guided endonuclease.

19. The method of claim 13, wherein said DNA construct further comprises at least one cassette encoding one or more guide RNAs.

20. The method of claim 11, further comprising:
c) selecting a plant that exhibits haploid induction phenotype when crossed to a non-inducer plant.

21. (canceled)

22. (canceled)

23. (canceled)

24. A method for transferring haploid induction effect to new lines comprising the steps of:
a) providing a first plant comprising at least one supernumerary chromosome, wherein the at least one supernumerary chromosome comprises at least one genetic element that can cause haploid induction;
b) crossing the first plant with a second plant of interest; and
c) recovering a third plant resultant from crossing the first plant and second plant, wherein the third plant comprises at least one supernumerary chromosome comprises at least one genetic element that can cause haploid induction.

25. The method of claim 16, wherein the supernumerary chromosome is a B chromosome.

26. The method of claim 17, wherein the B chromosome is selected from the group consisting of a corn B chromosome and a rye B chromosome.

27. The method of claim 16, wherein the at least one supernumerary chromosome is an artificially derived chromosome.

28. The method of claim 19, wherein the artificially derived chromosome is a truncated chromosome or a de novo generated chromosome.
